(19) United States
(12) Patent Application Publication
(10) Pub. No.: US 2024/0408288 A1
(43) Pub. Date: Dec. 12, 2024

(54) SYSTEM AND APPARATUS FOR THE TREATMENT OF MASTITIS

(71) Applicant: Jonathan Craig Gillan, Tempe, AZ (US)

(72) Inventor: Jonathan Craig Gillan, Tempe, AZ (US)

(21) Appl. No.: 18/736,279

(22) Filed: Jun. 6, 2024

Related U.S. Application Data
(60) Provisional application No. 63/471,427, filed on Jun. 6, 2023.

(51) Int. Cl. A61M 1/06

(52) U.S. Cl. CPC A61M 1/0697; A61M 1/064; A61M 2205/0216

(57) ABSTRACT

The present disclosure relates to an apparatus and methods for treating mastitis in lactating mothers by addressing clogged milk ducts. In some embodiments, the system includes an angled pump flange designed to work with a breast pump to facilitate the removal of ductal plugs that lead to mastitis. In some embodiments, the flange is structured to maintain substantial contact with the breast, allowing for manipulation such as pulling, pushing, and rotation, to dislodge plugs while a vacuum is applied. In some embodiments, the interior surface of the flange provides increased friction against the skin to resist relative motion during manipulation. In some embodiments, the flange's semi-rigid or rigid construction maintains its shape during use, enabling effective manipulation of the breast without losing connection. In some embodiments, the system may also include a breast pump with adjustable suction cycles to simulate a nursing baby and encourage milk flow.

### CROSS-REFERENCE TO RELATED APPLICATION

This application claims priority and the benefit of U.S. Provisional Pat. No. 6,317,427, filed Jun. 6, 2023, the entire contents of which are incorporated herein by reference.

### BACKGROUND

Breast pumps are used in hospital settings to harvest milk from new mothers that, for various reasons, are not able to bring baby to the breast and nurse naturally. A common reason for this separation between mother and infant is in the case of a premature baby that is not allowed outside of a controlled environment helping them to mature. Knowing that breastmilk has the nutrients and the antibodies their baby needs, these new mothers have no other option than to pump. Many of them have never even nursed before so the activity is even more foreign to them. Babies typically cat 8-10 times/day so pumping for a preemie is pretty much every 2-3 hours . . . around the clock. Lack of sleep, fatty meals (fast food is easy to sneak into a hospital by a caring partner) and a breast that is not accustomed to this relentless assault can easily lead to a fatty clump that gets stuck and causes the blockage of a milk duct which is referred to as a clogged duct. It is not uncommon for mothers to get clogged ducts in a hospital setting but having no access to baby, no skin-to-skin contact, no sensory ignition, etc. puts the mother of a preemie at an especially precarious position to perform in a mostly unnatural way.

Clogged ducts are blockages of a milk duct by a plug. Plugs are typically dried milk or clumps of milk fat found in the milk that gets stuck in a breast milk duct and creates a backup of milk in that duct. If left untreated a clog usually results in an onset of infection, a very painful and risky situation. FIG. 1 shows the structure of the lactation system of the human breast, and also shows a clogged duct and associated inflamed infected material according to some embodiments. The ducts travel in a relative straight direction towards the nipple and/or do not cross each other.

As the infection takes hold, mastitis sets in with fever, a fiery redness comes over the breast, and the patient begins to shiver. These symptoms are easily identified by medical professionals. Worry falls over the patient and the realization that antibiotics will need to be taken to fight the infection and the fear that they may not be able to feed their baby is emotionally draining and physically taxing. Depending on the seriousness of the infection, the patient may take days to recover with antibiotics or, in extreme cases, risk permanent stoppage of milk production. Any scenario where infection is in the breast is risky and stopping the infection from progressing is paramount. Mastitis, like all infection, can be life altering for mother and baby and can be very difficult to rebound from. When milk reservoirs dry up, the mother is forced to choose alternatives.

FIG. 2 shows another view of a clogged duct according to some embodiments. As shown in FIG. 2, milk forms in the lobules in the breast and flows through the ducts and out of the nipple, but one of the ducts has a small clog and is beginning to block the flow of the duct restricting it from draining according to some embodiments.

FIG. 3 illustrates the formation of a lump in the breast as a result of the clog according to some embodiments. As shown in FIG. 3, the clog continues to cause a backup of milk, as there is no place for the milk to go other than out through the duct. The affected lobule continues to secrete milk along with the other lobules (there is no way to stop it) and pressure starts to build. The lobule begins to swell, and a lump begins to form that is quickly and easily visible to the naked eye. Discomfort from the swelling begins almost immediately.

FIG. 4 shows the next stage in the formation of the lump according to some embodiments. As shown in FIG. 4, the plug is completely dry and is connected strongly enough to the walls of the duct that the pressure of the backed-up milk, the swelling of the lobule, and even gentle massage of the breast cannot push the plug out. The lump gets harder and more painful and, more importantly, the lobule is in jeopardy of infection setting in quickly.

The function of a human breast pump system in the current art is not to suck the milk out or mechanically extract the milk (as in a dairy setting), the job of the modern breast pump is to use a cycling suction function to stimulate the nipple and get the body to respond to the stimulation which instructs the breast to push the milk through the milk ducts and exit out of the nipple. Accordingly, prior art and claims concerning pumps and flanges (the only part of the pump that touches the pumping mother) are focused on what happens outside of the breast and are not capable of producing the results described herein.

In the prior art, most pump flange technology centers around a funnel that goes on the breast and a tunnel, or passageway, that allows the milk to be captured in a vessel. In the prior art, flanges are always made in a way that allows the breast to move freely inside the flange. However, there is currently no system involving the use of a breast pump that addresses mastitis happening on the inside of a breast.

FIG. 5 shows a prior art generic straight flange typically made from hard plastic with a slippery inside surface and a distinctive bend (change of direction) from the funnel to the tunnel. FIG. 6 shows a prior art angled flange of U.S. Patent Application Publication #US2006/0116632A1, which has one long side that remains relatively flat. U.S. Patent Application Publication #US2006/0116632A1 also discusses how the prior art is intended to allow relative motion between the flange and the skin (i.e., be slippery).

Currently there exists no effective care for mothers that are experiencing the onset of mastitis. Massage plus warming of the breast with hot packs, or cooling with cold packs, is common but lacks consistent results. Therefore, there is a need in the art to be able to both manipulate the breast and its internal structures while simultaneously stimulating the breast to dislodge a clog.

### SUMMARY

In some embodiments, the present disclosure provides an apparatus comprising one or more of an angled pump flange, a vacuum connection end, and a breast connection end. In some embodiments, the angled pump flange is configured to stimulate a nipple and instruct a breast to push milk through milk ducts and exit out of the nipple. In some embodiments, the vacuum connection end is coupled to the breast connection end. In some embodiments, the inside of the angled pump flange comprises an inner surface that includes a texture configured to resist relative motion between the inner surface and skin of the breast.

In some embodiments, the angled pump flange includes at least one side configured to run substantially straight along the side of a breast. In some embodiments, the angled pump flange comprises a semi-rigid structure flexible enough to enable the breast connection end to roll back toward the vacuum connection end. In some embodiments, the inside of the angled pump flange is rougher than the outside of the angled pump flange. In some embodiments, the semi-rigid structure of the angled pump flange is configured to substantially maintain its shape when moving the breast in one or more directions.

In some embodiments, the semi-rigid structure of the angled pump flange is configured to enable manipulation of a breast up to 6 pounds. In some embodiments, the semi-rigid structure of the angled pump flange comprises a funnel portion that is foldable over a tunnel portion. In some embodiments, the semi-rigid structure of the angled pump flange comprises a breast connection end that is rollable back toward the vacuum connection end. In some embodiments, the increased friction of the inner surface of the angled pump flange substantially prevents relative motion of skin between the breast and the flange. In some embodiments, the inner surface of the angled pump flange comprises one or more ripples configured to increase the ability of the funnel to maintain suction against the skin.

In some embodiments, a method for treating mastitis includes one or more steps listed herein. Some embodiments include a step of applying an angled pump flange to a breast of a lactating mother. Some embodiments include a step of positioning the angled pump flange to align with at least one milk duct of the breast. Some embodiments include a step of manipulating the breast with the angled pump flange while applying a vacuum to dislodge a plug in the milk duct.

Some embodiments include a step of adjusting the suction of a breast pump connected to the angled pump flange to a level sufficient to maintain contact with the breast during manipulation. Some embodiments include a step of rolling back a portion of the angled pump flange to facilitate application to the breast. In some embodiments, the manipulating step includes pulling on the angled pump flange to straighten and stretch the milk duct. In some embodiments, adjusting the suction includes selecting a vacuum pressure within a range of −70 mmHG to −150 mmHG. In some embodiments, adjusting the suction includes setting a breast pump to alternate between a long suction cycle and a rapid cycle to stimulate the nipple.

In some embodiments, rolling back a portion of the angled pump flange includes inverting a funnel portion of the angled pump flange to expose an inner gripping surface that has a higher friction coefficient than an outer surface of the angled pump flange. In some embodiments, rolling back a portion of the angled pump flange includes using the rolled back portion to draw a nipple into a tunnel portion of the flange. In some embodiments, pulling on the angled pump flange includes applying a force in a direction parallel to the at least one milk duct. In some embodiments, pulling on the angled pump flange includes applying a force in multiple directions to manipulate the breast and assist in dislodging the plug.

### DRAWING DESCRIPTIONS

FIG. 1 shows the structure of the lactation system of the human breast, and also shows a clogged duct and associated inflamed infected material according to some embodiments.

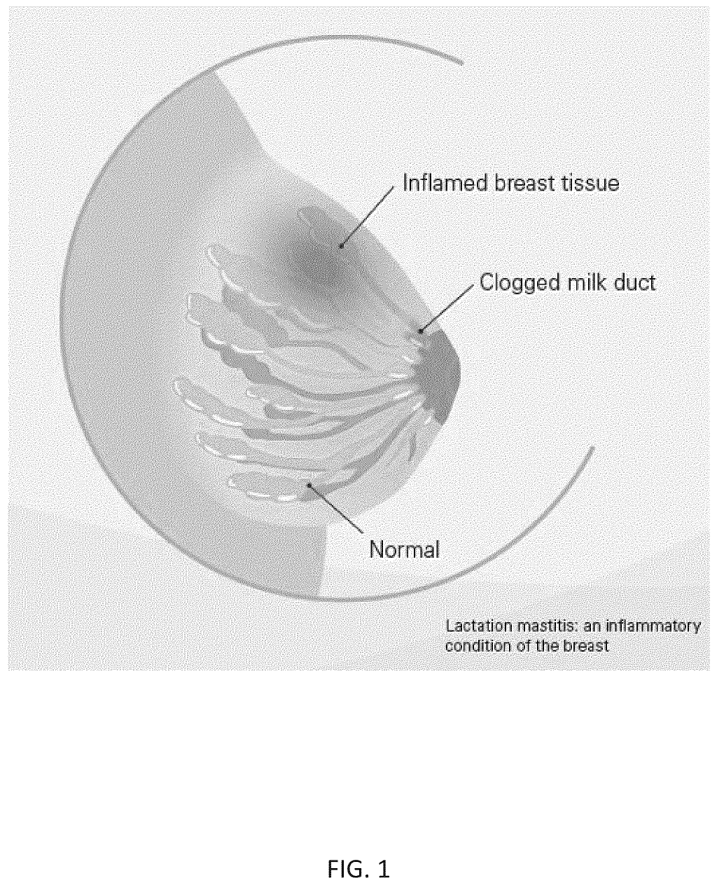

FIG. 2 shows another view of a clogged duct according to some embodiments.

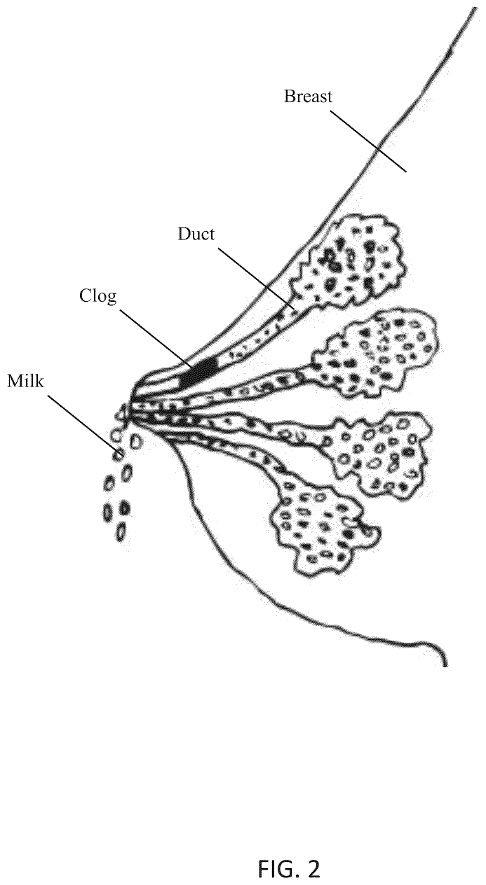

FIG. 3 illustrates the formation of a lump in the breast as a result of the clog according to some embodiments.

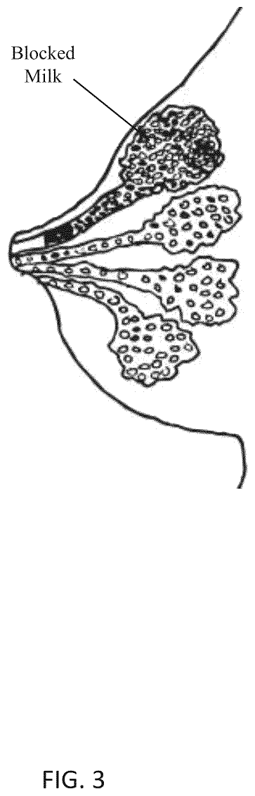

FIG. 4 shows the next stage in the formation of the lump according to some embodiments.

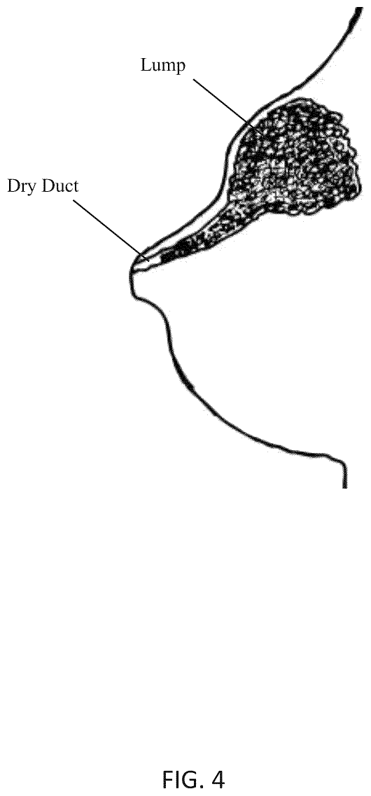

FIG. 5 shows a prior art generic straight flange typically made from hard plastic with a slippery inside surface and a distinctive bend (change of direction) from the funnel to the tunnel.

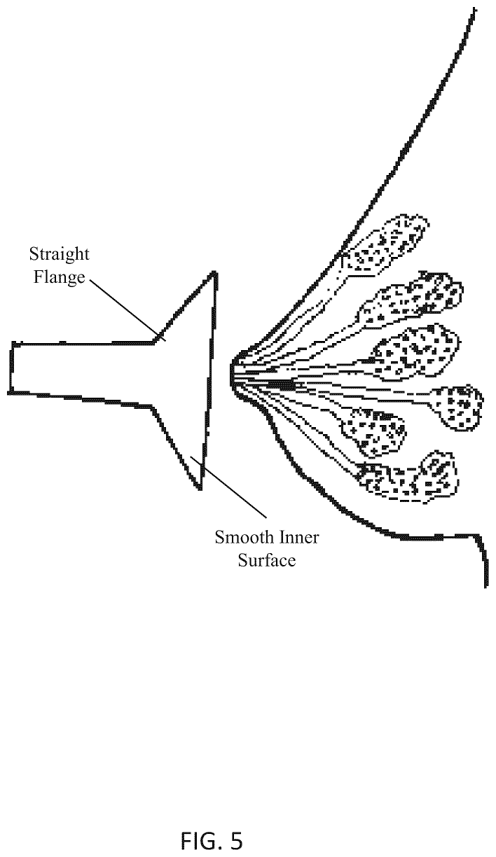

FIG. 6 shows a prior art angled flange which has one long side that remains relatively flat.

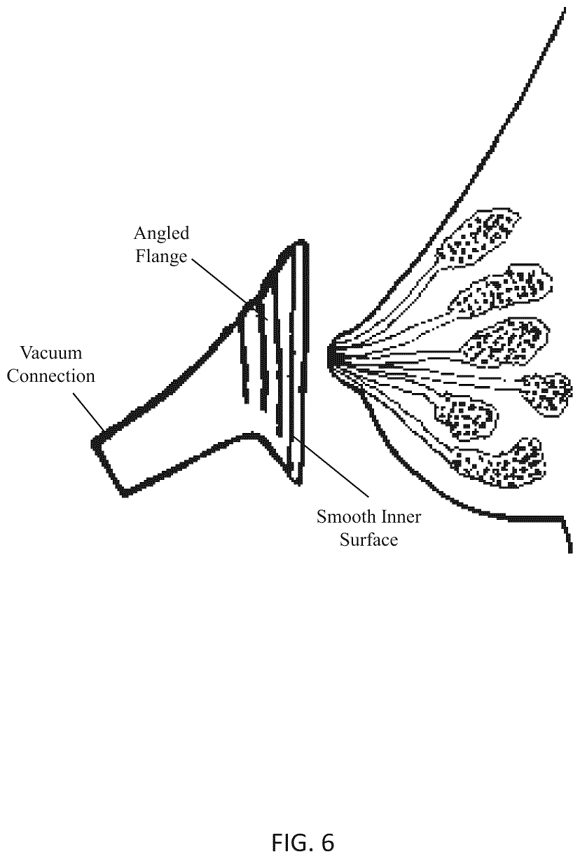

FIG. 7 shows some embodiments of the system including a pump outfitted with an angled pump flange of the system according to some embodiments.

FIG. 8 shows the angled flange applied to the breast a well as the applied direction of the inner surface grip on the skin according to some embodiments.

FIG. 9 illustrates movements as the technician pulls on the flange to straighten the duct and stretch the duct according to some embodiments.

FIG. 10 illustrates a step in a method of use where a technician rotates the flange relative to the breast up and begins to compress the duct against itself, attempting to loosen the clog according to some embodiments.

FIG. 11 illustrates the plug breaking away from the sides of the duct and the plug moving through the duct and being deposited in the bottle according to some embodiments.

FIG. 12 illustrates a non-limiting method of flange application that includes rolling back the funnel portion of the angled flange according to some embodiments.

### DETAILED DESCRIPTION

In some embodiments, the disclosure is directed to systems and methods for reversing the progression of Mastitis infection and inflammation from the breast of a lactating mother suffering from a clogged milk duct(s) by complete removal of the plug(s). In some embodiments, the system includes an angled pump flange. In some embodiments, the angle pump flange comprises at least one side (and/or surface quadrant) configured to run (substantially straight) along the side of a breast in a (substantially parallel) direction of one or more milk ducts.

In some embodiments, a first surface quadrant is longer than a second surface quadrant. In some embodiments, a first end the first surface quadrant extends further from a pump opening than a second end of a second surface quadrant. In some embodiments, the first surface quadrant comprises a top of the angled flange. In some embodiments, the second surface quadrant comprises a bottom of the flange.

In some embodiments, the angled flange comprises a third surface quadrant connecting the first surface quadrant and the second surface quadrant on one side. In some embodiments, the angled flange comprises a third surface quadrant connecting the first surface quadrant and the second surface quadrant on an opposite side. In some embodiments, both the third surface quadrant and the fourth surface quadrant extend from the pump opening at a distal end of the flange a substantially equal distance.

In some embodiments, the flange comprises a semi-rigid structure. In some embodiments, the semi-rigid structure is flexible enough to enable the breast connection end to roll back toward the vacuum connection end, such that the funnel portion is folded over the tunnel portion. In some embodiments, the flange comprises a rigid structure. In some embodiments, the rigid structure is rigid enough to prevent the breast connection end to roll back to where the funnel portion is folded over the tunnel portion.

In some embodiments, the inside of the flange is not smooth. In some embodiments, the inside of the flange is sticky and/or tacky to the point where the inner surface will provide increased friction against skin greater than the friction of a smooth surface, where the increased friction resists relative motion between the skin and the flange. In some embodiments, the flange comprises one or more ripples on the funnel area. In some embodiments, the one or more ripples are proximate the transition from the funnel area to the tunnel area. In some embodiments, the one or more ripples are in a ring configuration around the funnel area. In some embodiments, the one or more ripples comprise a raised portion on an outer surface of the funnel portion of the flange and/or a recessed portion on an inner surface of the funnel portion of the flange. In some embodiments, the funnel portion comprises three or more ripples, where the alternating patterns create a wave shape along an inner flange surface and/or outer flange surface. In some embodiments, the one or more ripples are configured to increase the ability of the funnel to maintain suction against the skin. In some embodiments, increasing the number of ripples increases the ability to maintain connection between the flange and the breast without increasing vacuum suction. In some embodiments, three or more ripples have been found to enable sufficient connection strength using a minimum vacuum pressure. In some embodiments, a single ripple requires more vacuum strength to maintain a connection than may be comfortable for a patient. In some embodiments, the vacuum connection portion of the flange comprise one or more ridges along the outer portion of the flange configured to increase compressive force against a vacuum connection on a pump.

In some embodiments, a manufacturing system and method includes manufacturing the angled flange in such a way that the surface of the inside of the flange is configured to create a bond between an internal surface of the flange (created by all four quadrants) and the surface of the skin strong enough for the breast to remain in contact with the flange when pulled by a technician in the direction of the flow of the ducts when a vacuum is applied without the flange losing connection with the breast. In some embodiments, the bond is strong enough to lift up to 10 pounds (e.g., a 6 pound breast) of skin against the force of gravity. In some embodiments, a method of use for the system includes steps to be taken by a technician that results in the dislodging of a plug in the breast and a release of milk from the breast lobule averting infection of the lobule.

In some embodiments, a method to remove clogs located in milk ducts of nursing mothers includes a step of utilizing an angled pump flange designed and manufactured to grip the breast and allow for breast manipulation (pulling, pushing, up, down, and side to side) without disconnecting when under (a cycled) vacuum so as to release the clog and terminate the onset of mastitis infection. In some embodiments, the inner surface of the flange comprises a roughened surface. In some embodiments, the system includes an adhesive configured to provide sufficient strength to maintain contact between the breast skin and the inner portion of the flange while under vacuum. In some embodiments, the adhesive is configured to enable a disconnect of the breast skin and inner portion of the flange when vacuum is not applied. In some embodiments, the adhesive is applied during the manufacturing process. In some embodiments, the system comprises a kit comprising a separate container for the adhesive.

In some embodiments, the system is configured to provide enough gripping force against breast skin to enable manipulation of the breast including pulling the breast away from the chest without disconnecting while a vacuum is applied, to break up a plug in a clogged duct. In some embodiments, a (breast) pump attached to the flange is used to continue to administer stimulation to the nipple with a suck/release cycle to simulate baby nursing movement. In some embodiments, the breast pump comprises a flange interface configured to secure a removable flange to the pump, where the flange is communicably connected to the vacuum. In some embodiments, the pump is configured to stimulate the nipple to encourage the body to produce a chemical (oxytocin) that causes the breast muscles to squeeze the lobules and push the milk into the ducts and out of the mother. In some embodiments, the system is configured to be compatible with a conventional breast pump (excluding a conventional flange). In some embodiments, the breast pump is a proprietary breast pump configured to apply a vacuum greater than a conventional breast pump.

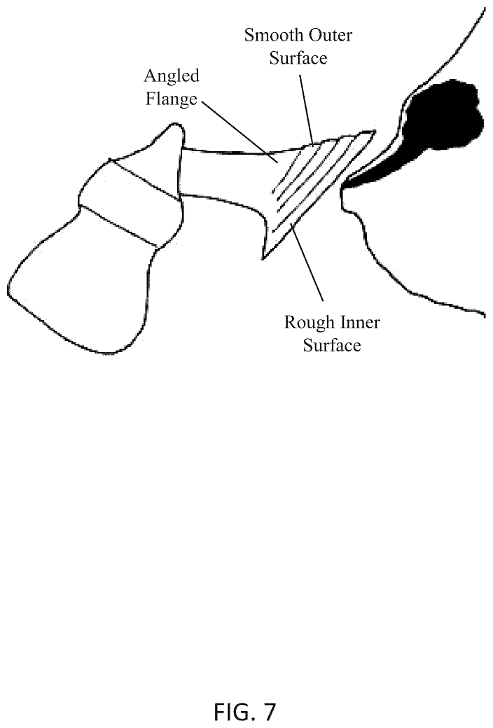

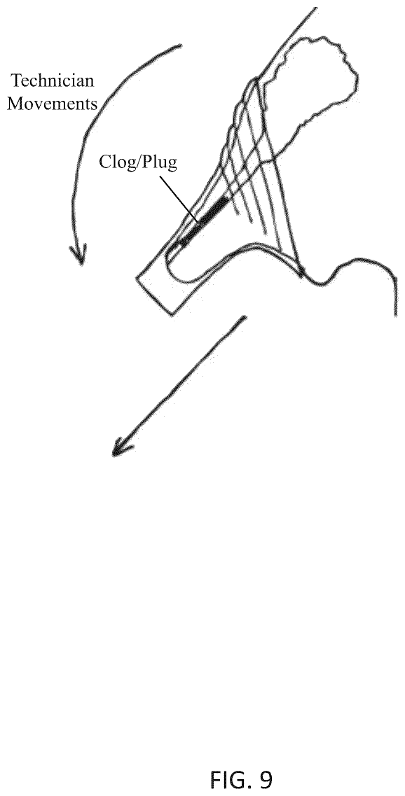

FIG. 7 shows some embodiments of the system including a pump outfitted with an angled pump flange according to some embodiments. In some embodiments, a step in a method of use includes positioning the top of the flange (i.e., the spot on the funnel furthest from the base and/or pump opening) in front of the visible lump. In some embodiments, a step in a method of use includes positioning the top of the flange in alignment with the visible lump. In some embodiments, a method of use includes rotating and/or positioning the flange to point towards any location on the breast where a hard lump has formed due to a plug. In some embodiments, a method of use for multiple hard lumps from multiple clogged ducts include treating each lump by positioning the angled flange as described herein for each lump and treating each lump independently. In some embodiments, a method of use includes positioning the longest side (i.e., top) of the angled flange to run parallel to the duct when the angled flange is applied to the breast as shown in FIG. 9. In some embodiments, one or more of these alignments are configured to dislodge a clog.

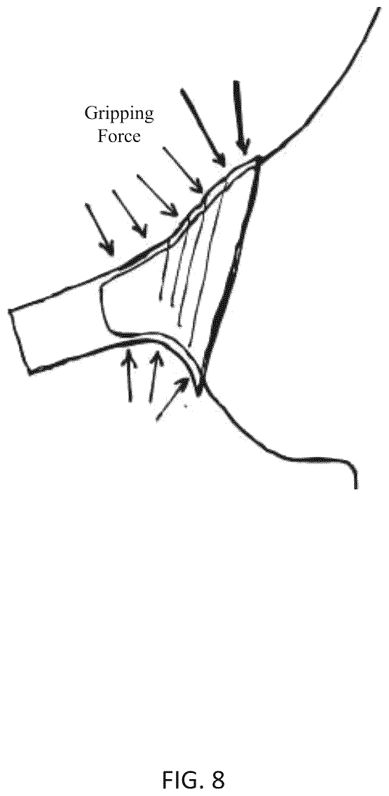

FIG. 8 shows the angled flange applied to the breast a well as the applied direction of the inner surface grip on the skin according to some embodiments. In some embodiments, the angled flange includes an internal surface configured to provide sufficient grip to maintain the relative position of the skin and angled flange when the angled flange and/or pump is moved. In some embodiments, the angled flange includes an internal surface configured to provide sufficient grip to maintain the nipple in substantially the same position within the angled flange when the angled flange moved under vacuum. In some embodiments, the angled flange includes an internal surface configured to provide sufficient grip to maintain the skin on the breast in contact with the internal surface in substantially the same position within the angled flange when the angled flange moved under vacuum. In some embodiments, the tunnel portion of the flange is wide enough to where a nipple can extend into the tunnel without touching the tunnel walls, such that the nipple can be extended by vacuum force as described herein according to a method of use.

In some embodiments, the system is configured to enable the suction of the pump to be adjusted. In some embodiments, although a pump may be able to provide enough suction to lift a breast, only an amount of suction sufficient to stretch the breast is needed to use the system and perform the methods described herein. In some embodiments, an acceptable range for suction includes −20 mmHG to −200 mmHG. In some embodiments, a range of −70 mmHG to −150 mmHG applied by the pump is sufficient to enable a technician to manipulate the breast without losing contact.

In some embodiments, the vacuum force is sufficient to pull a nipple up to 25 mm into the tunnel portion of the flange relative to a nipple position at no vacuum. In some embodiments, this allows a stretching of the nipple to release a clog that is located in the nipple area. In some embodiments, a technician may choose an angled flange of sufficient size to achieve enough tunnel space for the nipple to extend according to a method of use. In some embodiments, the pump comprises a stretch cycle configured to draw the nipple into the tunnel region. In some embodiments, the stretch cycle comprises an alternating vacuum cycle configured to enable the nipple to extend and/or contract into the tunnel portion.

In some embodiments, once the flange engulf's the end of the breast the special surface prep of the inside of the funnel grips on to the breast and does not release when manipulated. In some embodiments, the flange thickness and/or material properties have enough rigidity that it substantially maintains its shape when moving the breast in one or more directions, becoming in effect a handle for the medical technician giving the technician the ability to pull on the breast without the flange disconnecting from the breast. In some embodiments, a method of use include patient lifting their own breast to an orientation specified by the technician while the technician applies one or more techniques described herein. In some embodiments, this allows for a lower vacuum pressure to be applied.

In some embodiments, in a non-limiting example, maintaining its shape includes a change in relative position of plane parallel to the pump opening and a plane parallel to the skin opening being less than an angle of 15 degrees. In some embodiments, in a non-limiting example, maintaining its shape includes manipulation of a breast up to 6 pounds. In some embodiments, the system is configured to provide enough suction force and/or gripping force to lift a breast up to 6 pounds. In some embodiments, the system is configured to enable a user to adjust vacuum pressure until enough gripping force is achieved to manipulate a breast. In some embodiments, the system is configured to provide enough vacuum pressure to at least partially pull the nipple into the flange when under vacuum (e.g., 5 mm toward the pump opening after the vacuum is applied).

FIG. 9 illustrates movements as the technician pulls on the flange to straighten the duct and stretch it according to some embodiments. In some embodiments, the system is configured to allow the technician to pull down and away from the lump, stretching the the duct and starting the process of breaking the connection between the plug and the walls of the duct. In some embodiments, the stretching makes the duct a more malleable to allow the clog to pass. In some embodiments, applying the side of the flange parallel to the direction of the duct is configured to keep the duct straight when pulling in any direction. In some embodiments, a prior art flange does not have one long side like the angled flange of the system described herein. In some embodiments, the long side comprises the extended portion of the flange created by the angle of the flange opening relative to the tunnel opening.

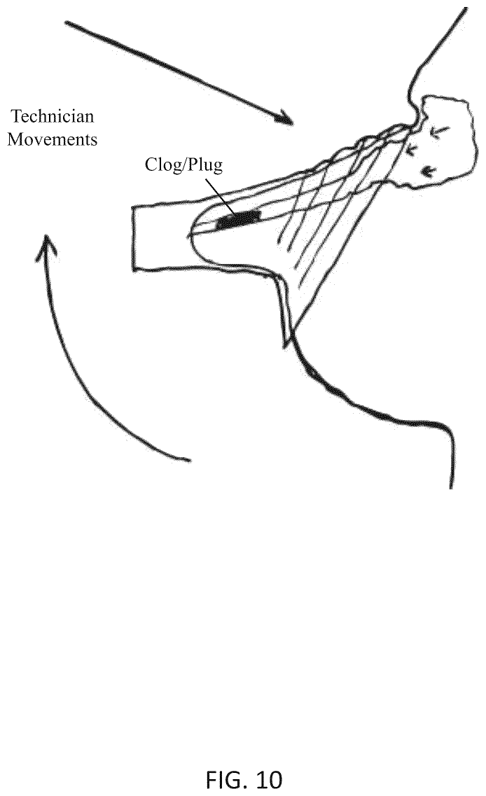

FIG. 10 illustrates a step in a method of use where a technician rotates the flange relative to the breast up and begins to compress the duct against itself, attempting to loosen the clog according to some embodiments. In some embodiments, as the flange is rotated back and towards the lump the top of the flange is configured to apply inward pressure on the lump and the entire duct to compress the plug and/or increase internal pressure. In some embodiments, the system is configured to enable the application of the inward pressure using an end section of the long portion of the flange while the pump is applying a vacuum and/or a cycled vacuum pressure. In some embodiments, applying a vacuum includes varying pressure and/or inducing vibration to stimulate the nipple.

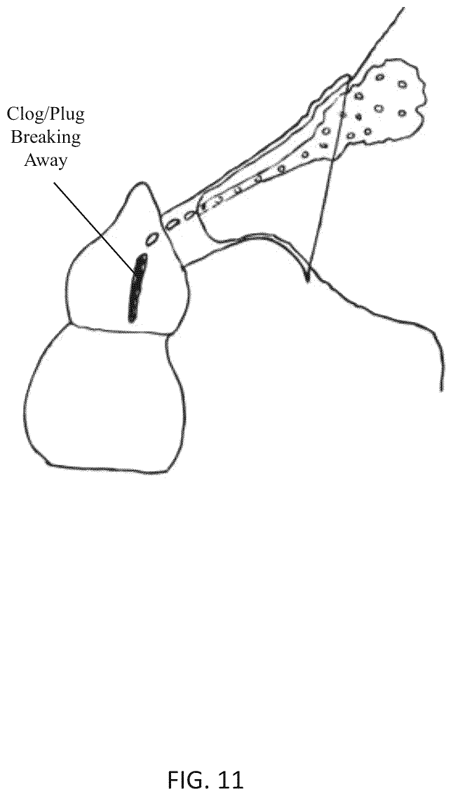

FIG. 11 illustrates the plug breaking away from the sides of the duct and the plug moving through the duct and being deposited in the bottle according to some embodiments. In some embodiments, once the clog has been released all of the backed-up milk comes rushing out behind the plug.

In some embodiments, a method of use includes one or more steps described herein. In some embodiments, one or more steps include a user attaching the angled flange to a pump and turning the pump on. In some embodiments, one or more steps include the user adjusting a pump setting to establish a long suction cycle. In some embodiments, one or more steps include a user identifying the location of the lump. In some embodiments, one or more steps include using the location of the lump to determine the positioning of the flange as it is applied to the breast. In some embodiments, one or more steps include positioning the flange with the long side of the flange following parallel the direction of the duct and or parallel to a line between the nipple and the lump.

In some embodiments, one or more steps include the user (e.g., technician) rolling back the funnel of the angled silicone flange (i.e., turning the funnel part inside out) and holding the flange very close to the breast (e.g., between 1 mm and 10 mm) without actually touching the breast.

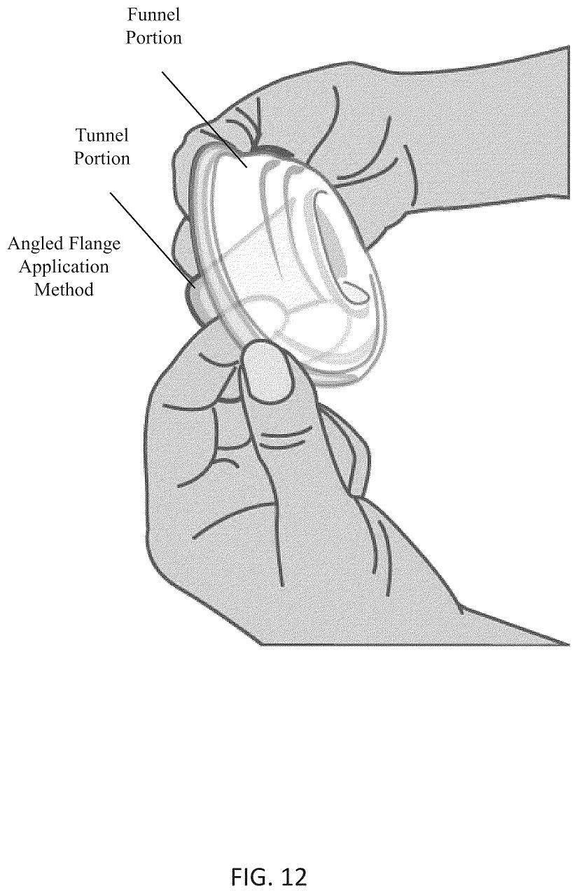

FIG. 12 illustrates a non-limiting method of flange application that includes a step of rolling back the funnel portion of the angled flange according to some embodiments. In some embodiments, one or more steps include allowing the vacuum pressure from the pump suction to pull the nipple towards the opening of the tunnel (e.g., like how when a vacuum cleaner gets close to something and that something starts to move towards the vacuum cleaner wand) and/or pull the areola up to and in contact with the surface of the flange. In some embodiments, one or more steps include the technician allowing the funnel portion of the flange to fold back to its natural state in a manner that collapses around and encompasses the end of the breast making sure the nipple is positioned inside the tunnel part of the flange. In some embodiments, a step includes positioning the longest portion of the elongated flange on the top portion of the breast (relative to the floor).

In some embodiments, the funnel portion of the angled flange is configured to fold back to a point where the outer surface of the funnel portion touches the tunnel portion adjacent the vacuum connection end. In some embodiments, the tunnel portion is configured to maintain a substantially same shape when the tunnel portion outer surface touches the funnel portion outer surface when rolled back. In some embodiments, the tunnel portion is configured not to collapse when the funnel portion is rolled back. In some embodiments, the tunnel portion is configured to maintain a substantially same flowrate and/or or vacuum force when the tunnel portion outer surface is in contact with the funnel portion outer surface. By not collapsing, the angled flange can maintain a vacuum force while being rolled back which is an improvement over the prior art.

In some embodiments, the funnel portion is configured to return to an original shape after being rolled back to touch the tunnel portion. In some embodiments, the funnel portion is configured to return to an original shape after being rolled back multiple times. In some embodiments, the funnel portion is configured to return to an original shape after being rolled back more than 10 times, which prior art flanges cannot do without significant deformation or failure.

In some embodiments, one or more steps include the user adjusting the pump to a more rapid cycle designed to stimulate the nipple. In some embodiments, one or more steps include a user alternating back to long suction cycles. In some embodiments, one or more steps include alternating between long and rapid cycles as needed for manipulation of the duct. In some embodiments, one or more steps include the user holding the flange and using it like a handle to stretch the breast in the same direction as the established duct line. In some embodiments, one or more steps include then pulling perpendicular to the right, then the left, then back towards the breast. In some embodiments, one or more steps include pulling while the pump continues to stimulate the nipple.

In some embodiments, a pump suction cycle includes a rapid cycle configured to stimulate the nipple. In some embodiments, the rapid cycle includes a cycle time of between ¼ to ¾ (e.g., ½) seconds. In some embodiments, the pump suction cycle includes an extended cycle configured to facilitate expression. In some embodiments, the extended cycle includes a cycle time between 1-2 seconds. In some embodiments, the pump is configured to enable a user to set a cycle time between ⅛ of a second to 3 seconds. In some embodiments, the pump is configured to maintain a vacuum pressure between −50 mmHG and −100 mmHG during one or more cycles. In some embodiments, a vacuum pressure between −50 mmHG and −70 mmHG has been found to provide a good balance of effectiveness and patient comfort.

In some embodiments, after a few minutes of manipulation the clog releases, a (e.g., +1 inch) hardened spaghetti looking piece of dried milk comes out and the backed-up milk behind the clog comes rushing out. As mentioned before, current standards of care to remove clogs from ducts does not include the use of a pump to maintain stimulation and/or substantially continuous contact of the nipple and/or breast tissue while other techniques are attempted. In the prior art, typical pump flange shapes and hard and/or smooth plastic surfaces are designed to allow breast tissue to move freely and easily across the surface of the flange. Therefore, if manipulation of the breast is attempted with a prior art flange the connection between the flange and the breast will break and the flange will disconnect from the breast and no longer stimulate the nipple.

In some embodiments, one or more steps include soaking the breast. In some embodiments, one or more steps include breaking down larger clumps by kneading (stripping) the nipple. In some embodiments, one or more steps include using the suction from the pump to stimulate the nipple keeping the letdown response going and then using the flange and its' ability to hold on to the breast well enough to stretch the breast in a parallel direction of the duct with the clog (e.g., stretch and release, stretch and release) until the clog breaks away from the inside walls of the duct and can flow downstream and out of the nipple. In some embodiments, the pulling of the breast in the direction of the duct and subsequent manipulation while the suction function of the pump continues to stimulate the nipple is completely novel to the disciplined care of providers assisting mothers facing the onset of mastitis.

In some embodiments, the system includes a method of manufacture for a breast pump flange. In some embodiments, a system and method of manufacture includes providing a flange mold configured to create a grippy and/or sticky surface on the inner portion of the flange where skin contact occurs. In some embodiments, the system includes the mold.

In some embodiments, the process for making the inside of the flange sticky enough to grip the breast tissue and hold on to it has two major components: polish of the injection mold surface and hardness (e.g., Shore) of the angled flange material. In some embodiments, the material is silicone. In some embodiments, the system includes one or more of a plurality of flange sizes to accommodate different breast sizes and/or breast weights. In some embodiments, each size includes a different wall thickness. In some embodiments, each of the one or more of the plurality of flange sizes are stiff enough to not collapse inward due to the suction created by the pump. In some embodiments, each of the one or more of the plurality of flange sizes include an inside surface soft enough to grip the breast tissue and hold on at a greater strength than the suction provides alone. In some embodiments, each of the one or more of the plurality of flange sizes includes a shore between 40-60. In some embodiments, a thicker flange wall correlates to a lower shore. In some embodiments, a 0.10 inch wall results in a hardness of 60 shore. In some embodiments, a 0.14 in wall thickness results in a hardness of 40 shore. In some embodiments, the shore hardness depends on product thickness. In some embodiments, method of manufacture includes a mold with the portion forming the inner portion of the flange polished the surface to an SPI (Society of Plastic Industry) A2 finish, as non-limiting example. In some embodiments, the inner portion of the angled flange and/or mold includes an SIP A1-A3 finish. In some embodiments, a portion of the mold forming the inner portion of the flange includes a mirror like finish. In some embodiments, the mold finish and the softness of the surface work together with the shape of the opening to the flange to physically grab on and hold on to the breast tissue.

The industry standard for surface of silicone flanges or inserts of the prior art is a very low polish or bead blast (frosted) seeking a soft feel surface that allows free movement of the tissue it is in contact with. The prior art flanges are usually opaque because of nature of the surface. The uniqueness of the system described herein is rooted in the attempt to do the unthinkable in the industry: grab on and hold on to the tissue. The prior art flanges want relative movement between the skin and the flange to enable milk flow in a non-clogged duct. The idea of not letting tissue move when the nipple is getting pulled on was not something anyone wanted to even try, and therefore the prior art teaches away from the system, apparatus, and methods described herein.

The entirety of Appendices A, B, and C, in Provisional Application No. 63/471,427 are incorporated by reference herein. Appendix A includes a technical data sheet for a LIM6050 liquid silicone rubber suitable for an injection molding process for the angled flange according to some embodiments. Appendix B includes a technical data sheet for a Silopren LSR 2640 liquid silicone rubber suitable for an injection molding process for the angled flange according to some embodiments. Appendix C includes a technical data sheet for a Silopren LSR 2650 liquid silicone rubber suitable for an injection molding process for the angled flange according to some embodiments.

The subject matter described herein are directed to technological improvements to the treatment of mastitis by enabling a breast to be stretched while manipulating internal structures of the breast with the same device applying a vacuum and/or gripping force to the breast. The systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. All flowcharts presented herein represent computer implemented steps and/or are visual representations of algorithms implemented by the system. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. An apparatus comprising:
an angled pump flange,
a vacuum connection end, and
a breast connection end;
wherein the angled pump flange is configured to stimulate a nipple and instruct a breast to push milk through milk ducts and exit out of the nipple;
wherein the vacuum connection end is coupled to the breast connection end; and
wherein the inside of the angled pump flange comprises an inner surface that includes a texture configured to resist relative motion between the inner surface and skin of the breast.

2. The apparatus of claim 1,
wherein a tunnel portion of the angled pump flange adjacent the vacuum connection end is configured not to collapse when a funnel portion of the angled pump flange is rolled back.

3. The apparatus of claim 1,
wherein the angled pump flange comprises a semi-rigid structure flexible enough to enable the breast connection end to roll back toward the vacuum connection end.

4. The apparatus of claim 1,
wherein the inside of the angled pump flange is rougher than the outside of the angled pump flange.

5. The apparatus of claim 3,
wherein the semi-rigid structure of the angled pump flange is configured to substantially maintain its shape when moving the breast in one or more directions.

6. The apparatus of claim 5,
wherein the semi-rigid structure of the angled pump flange is configured to enable manipulation of a breast up to 6 pounds.

7. The apparatus of claim 3,
wherein the semi-rigid structure of the angled pump flange comprises a funnel portion that is foldable over a tunnel portion.

8. The apparatus of claim 3,
wherein the semi-rigid structure of the angled pump flange comprises a breast connection end that is rollable back toward the vacuum connection end.

9. The apparatus of claim 4,
wherein the increased friction of the inner surface of the angled pump flange substantially prevent relative motion of skin between the breast and the flange.

10. The apparatus of claim 4,
wherein the inner surface of the angled pump flange comprises one or more ripples configured to increase the ability of the funnel to maintain suction against the skin.

11. A method for treating mastitis, comprising:
applying an angled pump flange to a breast of a lactating mother;
positioning the angled pump flange to align with at least one milk duct of the breast; and
manipulating the breast with the angled pump flange while applying a vacuum to dislodge a plug in the milk duct.

12. The method of claim 11, further comprising:
adjusting the suction of a breast pump connected to the angled pump flange to a level sufficient to maintain contact with the breast during manipulation.

13. The method of claim 11, further comprising:
rolling back a portion of the angled pump flange to facilitate application to the breast.

14. The method of claim 11, wherein the manipulating step includes:
pulling on the angled pump flange to straighten and stretch the milk duct.

15. The method of claim 12, wherein adjusting the suction includes:
selecting a vacuum pressure within a range of −70 mmHG to −150 mmHG.

16. The method of claim 12, wherein adjusting the suction includes:
setting a breast pump to alternate between a long suction cycle and a rapid cycle to stimulate the nipple.

17. The method of claim 13,
wherein rolling back a portion of the angled pump flange includes:
inverting a funnel portion of the angled pump flange to expose an inner gripping surface that has a higher friction coefficient than an outer surface of the angled pump flange.

18. The method of claim 13, wherein rolling back a portion of the angled pump flange includes:
using the rolled back portion to draw a nipple into a tunnel portion of the flange.

19. The method of claim 14, wherein pulling on the angled pump flange includes:
applying a force in a direction parallel to the at least one milk duct.

20. The method of claim 14, wherein pulling on the angled pump flange includes:
applying a force in multiple directions to manipulate the breast and assist in dislodging the plug.
